(19) United States
(12) Patent Application Publication
(10) Pub. No.: US 2020/0138624 A1
(43) Pub. Date: May 7, 2020

(54) FLEXIBLE THERMAL DEVICE

(71) Applicant: Joe TOLMAN, Keene, NH (US)

(72) Inventor: Joe TOLMAN, Keene, NH (US)

(21) Appl. No.: 16/660,526

(22) Filed: Oct. 22, 2019

Related U.S. Application Data
(60) Provisional application No. 62/756,675, filed on Nov. 7, 2018.

(51) Int. Cl. A61F 7/02; A61F 7/10

(52) U.S. Cl. CPC A61F 7/02; A61F 7/10; A61F 2007/108; A61F 2007/0225; A61F 2007/0295; A61F 2007/0219

(57) ABSTRACT

Flexible thermal devices comprising an elastic fabric ring having an inner fabric layer and an outer fabric layer and first terminal circumference at a first edge and a second terminal circumference at an opposite second edge, a plurality of panels defined between the inner fabric layer and the outer fabric layer creating a first panel row, and a plurality of gel cells being retained in the plurality of panels in the panel row, wherein the panels are bordered by stretch stitches joining the inner fabric layer and the outer fabric layer, and methods of treatment using the Flexible thermal devices.

### CROSS REFERENCE TO RELATED APPLICATIONS/PRIORITY

The present invention claims priority to United States Provisional Patent Application No. 62/756,676 filed Nov. 7, 2018, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### TECHNICAL FIELD

The present disclosure relates, generally, to a flexible thermal device. More particularly, the present disclosure relates to a flexible thermal device having a plurality of gel cells for heating or cooling an object or body.

### BACKGROUND

The use of cold or heat therapy has long been known in the medical field. Cold therapy may be used to treat certain limb injuries, such as sprained or strained arm or leg muscles, or injuries to joints. Heat therapy may be used to warm or limber muscles by increasing blood flow. A number of products may be used to provide heat or cold therapy. For example, gel-based hot or cold packs, are reusable but require the user to supply external energy before use, e.g., heat from a microwave oven or cold from a freezer. Generally, the gel packs are retained over the body part by holding the gel packs in hands or by using one or more straps fastening the gel packs to the body part. However, in such cases, the heat transfer between the body part and the gel packs is not efficient and also may not be evenly distributed.

### SUMMARY OF THE INVENTION

Wherefore, it is an object of an embodiment of the present invention to overcome the above-mentioned shortcomings and drawbacks associated with the current technology.

The invention relates to methods and flexible thermal devices comprising an elastic fabric ring having an inner fabric layer and an outer fabric layer and first terminal circumference at a first edge and a second terminal circumference at an opposite second edge, a plurality of panels defined between the inner fabric layer and the outer fabric layer creating a first panel row, and a plurality of gel cells being retained in the plurality of panels in the panel row, wherein the panels are bordered by stretch stitches joining the inner fabric layer and the outer fabric layer. According to a further embodiment the inner fabric layer and the outer fabric layer are formed of fabric that is elastic in four different directions. According to a further embodiment the inner fabric layer and the outer fabric layer are formed of fabric that can be stretched 25% longer than an unstretched length, and returns to the original length when released. According to a further embodiment the first panel row has at least four panels. According to a further embodiment a second panel row is adjacent to the first panel row. According to a further embodiment the second panel row is adjacent to the first panel row and spacing the first panel row from the second terminal circumference. According to a further embodiment a third panel row is adjacent to the second panel row, wherein the second panel row spaces the first panel row from the third panel row. According to a further embodiment the first, second, and third panel row each contain six panels. According to a further embodiment the first panel row contains an even number of panels. According to a further embodiment each panel has only one gel cell retained within. According to a further embodiment the stretch stitch is one of a Triple Straight Stitch, a Zigzag Stitch, a Three Step Zigzag stitch, a Lightning Bolt Stitch, a Honeycomb Stitch, an Overlock/Overedge Stitch, a Feather Stitch, a twin needle stitch, blind hem stretch stitch, and the stitch is with substantially non-elastic thread. According to a further embodiment the stretch stitch is a strait stitch with elastic thread. According to a further embodiment a first elastic cord is attached to the first terminal circumference and a second elastic cord is attached to the second terminal circumference. According to a further embodiment the gel cells contain one of diethylene glycol, ethylene glycol, hydroxyethyl cellulose, sodium polyacrylate, and vinyl-coated silica gel. According to a further embodiment the gel cells freeze solid at 0 degrees Celsius. According to a further embodiment the gel cells measure between 1 and 3 inches in width, 2 and 8 inches in height, and between 0.02 and 1 inch in thickness. According to a further embodiment adjacent gel cells are spaced between 0.2 and 0.8 inches from one another. According to a further embodiment the inner fabric layer and an outer fabric layer are one of spandex or spandex blended with a second fabric. According to a further embodiment the first and the second terminal circumference have a relaxed diameter 5% to 15% smaller than a diameter of the device at a mid-point between the first and the second terminal circumference.

The invention further relates to methods and flexible thermal devices comprising an elastic fabric ring having an inner fabric layer and an outer fabric layer and first terminal circumference at a first edge and a second terminal circumference at an opposite second edge; a plurality of panels defined between the inner fabric layer and the outer fabric layer creating a first panel row, the panels being bordered by stretch stitches joining the inner fabric layer and the outer fabric layer; a second panel row adjacent to the first panel row, the second panel row being adjacent to the first panel row and spacing the first panel row from the second terminal circumference; the first and the second panel row each contain six panels; a plurality of gel cells being retained in the plurality of panels in the first and the second panel row, with each panel having only one gel cell retained within; the gel cells measure between 1 and 3 inches in width, 2 and 8 inches in height, and between 0.02 and 1 inch in thickness; the inner fabric layer and the outer fabric layer are formed of fabric that is elastic in four different directions; the inner fabric layer and the outer fabric layer are formed of fabric that can be stretched 25% longer than an unstretched length in four directions, and returns to the original length when released; the stretch stitch being one of a Triple Straight Stitch, a Zigzag Stitch, a Three Step Zigzag stitch, a Lightning Bolt Stitch, a Honeycomb Stitch, an Overlock/Overedge Stitch, a Feather Stitch, a twin needle stitch, blind hem stretch stitch, and the stitch is with substantially non-elastic thread or being a strait stitch with substantially elastic thread, or some combination thereof; a first elastic cord being attached to the first terminal circumference and a second elastic cord being attached to the second terminal circumference; the gel cells containing one of diethylene glycol, ethylene glycol, hydroxyethyl cellulose, sodium polyacrylate, and vinyl-coated silica gel, or some combination thereof; the gel cells freezing solid when left for 24 hours at one of 2 degrees Celsius, 0 degrees Celsius, −5 degrees Celsius, and −10 degrees Celsius; adjacent gel cells being spaced between 0.2 and 0.8 inches from one another; the inner fabric layer and an outer fabric layer are one of spandex or spandex blended with a second fabric; and the first and the second terminal circumference having a relaxed diameter 5% to 15% smaller than a diameter of the device at a mid-point between the first and the second terminal circumference.

The invention is also related to methods of treating an injury by inserting an injured body part within the flexible thermal device, wherein the gel cells of the flexible thermal device are one of above 38, 40, 42, 45, or 48 degrees Celsius and below 9.5, 5, 2, or 0 degrees Celsius for a period of at least 15 minutes.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that though the accompanying drawings are to scale for one embodiment, the emphasis is instead placed on illustrating the principles of the invention. The invention will now be described, by way of example, with reference to the accompanying drawings in which:

FIG. 1 illustrates a flexible thermal device disposed over an arm of a wearer, in accordance with an embodiment of the disclosure;

FIG. 2 illustrates a perspective view of the flexible thermal device of FIG. 1, in accordance with an embodiment of the disclosure;

FIG. 3 illustrates a top sectional view of the flexible thermal device; in accordance with an embodiment of the disclosure;

FIG. 4 illustrates a front view of the flexible thermal device in a flat-out state to more clearly show the construction of the flexible thermal device, in accordance with an embodiment of the disclosure;

FIG. 5 illustrates a front view of a gel cell of the flexible thermal device; in accordance with an embodiment of the disclosure; and

FIG. 6 illustrates a front view of a flexible thermal device in a flat-out state to more clearly show the construction of the flexible thermal device, in accordance with an alternative embodiment of the disclosure.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

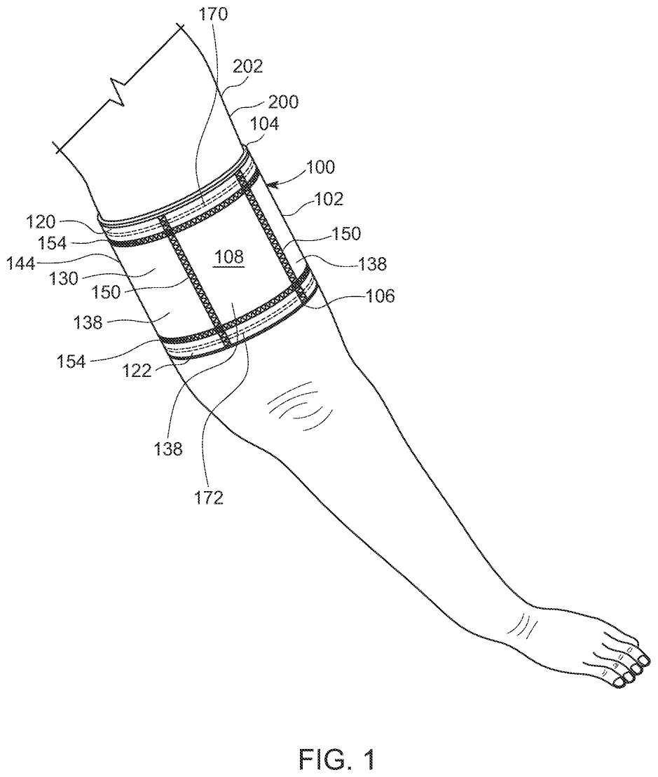

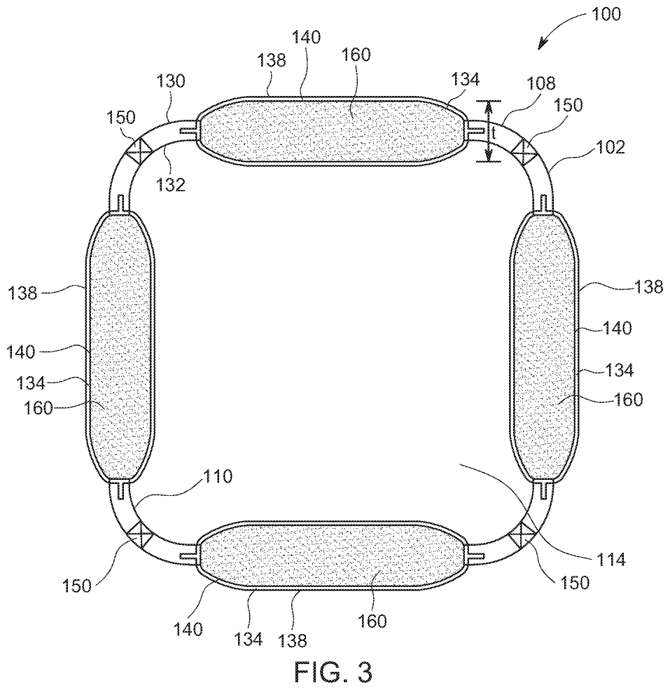

Turning now to FIGS. 1-6, a brief description concerning the various components of the present invention will now be briefly discussed. Referring to FIG. 1, a flexible thermal device (hereinafter referred to as a device 100) worn over a body part 200, such as an arm 202, of a person is shown. The device 100 is adapted to provide heating or cooling to the body part 200 of the wearer. Although the device 100 is shown as a sleeve worn by a person, it may be appreciated that the device 100 may be disposed over an article, such as, but not limited to, a bottle, a container, or any such article suitable for storing liquids or solid. Referring to FIGS. 1 to 4, the device 100 may include a circular configuration, and may include an elastic fabric ring 102 having a first edge 104 to a second edge 106, an outer surface 108, an inner surface 110 defining an elongated cavity 114 extending from the first edge 104 to the second edge 106 and adapted to receive the body part 200 or the article. The first edge 104 defines a first access opening 116 of the elongated cavity 114, while the second edge 106 defines a second access opening 118 of the elongated cavity 114. Further, the elastic fabric ring 102 includes a first terminal circumference 120 defined at the first edge 104 and a second terminal circumference 122 defined at the second edge 106, an outer fabric layer 130 defining the outer surface 108 and an inner fabric layer 132 disposed opposite and facing the outer fabric layer 130 and defining a plurality of pockets 134 (best shown in FIG. 3) therebetween. The inner fabric layer 132 defines the inner surface 110 of the elastic fabric ring 102 and is adapted to abut the body part 200 or the article.

In an embodiment, the inner fabric layer 132 is formed of a stretchable or elastic fabric that may stretch, for example, around 10%, 15%, 25%, 35%, or 50% longer than an unstretched length (i.e. an original length). In an embodiment, the elastic fabric may be extendible in one or more directions, for example, in four directions. Similarly, the outer fabric layer 130 is formed of a stretchable or elastic fabric that may stretch for example, around 10%, 15%, 25%, 35%, or 50% longer than an unstretched length (i.e. an original length). In an embodiment, the elastic fabric may be extendible in one or more directions, for example, in four directions. The term “elastic” is used herein includes any material which, upon application of a biasing force, is stretchable over 15% of an original or unstretched length, and which will recover to its original elongation upon release of the stretching, elongating force. As used herein, the term “nonelastic” includes any material which does not fall within the definition of “elastic,” above. In an embodiment, the inner fabric layer 132 and/or the outer fabric layer 130 may include spandex. Alternatively, the inner fabric layer 132 and/or the outer fabric layer 130 include spandex blended/mixed with a fabric made of another material (i.e. second fabric).

The device 100 further includes a plurality of panels 138 and a plurality of gel cells 140 (best shown in FIGS. 3, 4, and 5) disposed or retained inside the plurality of panels 138. The plurality of panels 138 are arranged in one or more panel rows and are defined between the inner fabric layer 132 and the outer fabric layer 130. In an embodiment, the plurality of panels 138 is defined as the plurality of pockets 134. In an exemplary embodiment, as shown in FIGS. 1 to 4, the device 100 may include a single panel row, for example, a first panel row 144, having four panels 138. Although the first panel row 144 is shown and contemplated to include four panels 138, it may be envisioned that the first panel row 144 may include any number of panels. In an exemplary embodiment the first panel row 144 may include an even number of panels. The even number of panels may facilitate a substantially flat positioning of the device 100 on a surface when the device 100 is laid on its side. In an embodiment, as shown, the panels 138 may be arrayed circularly around a central axis 146 and may be separated from adjacent panels by a plurality of stretch stitches 150 (hereinafter referred to as the plurality of first stretch stitches 150). As shown, each first stretch stitch 150 may extend longitudinally from the first edge 104 to the second edge 106 and may join/attach the inner fabric layer 132 to the outer fabric layer 130 together. Further, the device 100 may include a plurality of stretch stitches 154 (hereinafter referred to as the plurality of second stretch stitches 154) disposed proximate to the edges 104, 106, and extending circularly around the central axis 146. Further, the second stretch stitches 154 intersect with the first stretch stitches 150. In this manner, the panels 138 may be bordered by stretch stitches 150, 154. In an embodiment, each first stretch stitch 150 and/or each second stretch stitch 154 may be a strait stitch. In such a case, each first stretch stitch 150 and each second stretch stitch 154 may be formed using one or more elastic threads for facilitating a stretching of the first stretch stitch 150 and/or the second stretch stitch 154 when the device 100 is stretched/elongated from an original length (relaxed length). In an embodiment, the one or more elastic threads may stretch twenty five percent more than its original dimensions. Alternatively, each first stretch stitch 150 and/or each second stretch stitch 154 may be formed using substantially non-elastic threads. In such a case, the first stretch stitch 150 and/or the second stretch stitch 154 may be one of a Triple Straight Stitch, a Zigzag Stitch, a Three Step Zigzag stitch, a Lightning Bolt Stitch, a Honeycomb Stitch, an Overlock/Overedge Stitch, a Feather Stitch, a twin needle stitch, or a blind hem stretch stitch.

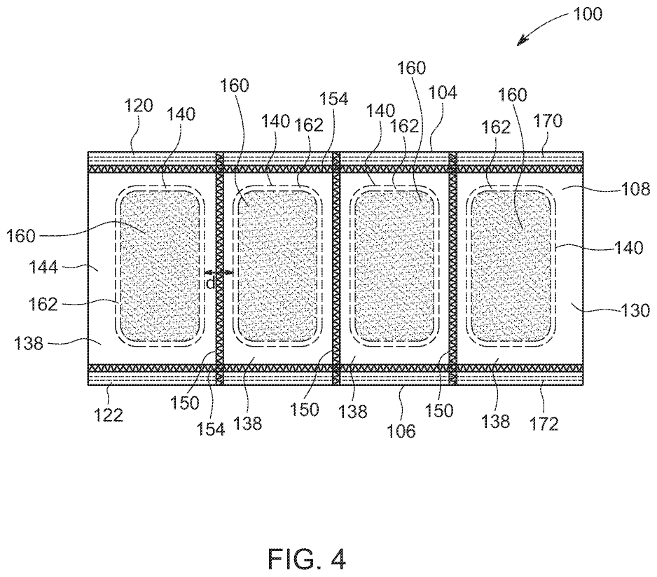

Further, each of the plurality of gel cells 140 facilitates an absorption/extraction of heat from the body part 200 and/or are adapted to provide heat to the body part 200. In an embodiment, each panel 138 may include a single gel cell 140. Although a single gel cell 140 is contemplated to be retained inside a single panel 138, it may be appreciated more than one gel cells 140 may be disposed inside each panel 138. Further, as shown, each gel cell 140 may be separated from an adjacent gel cell by a distance ‘d’ (best shown in FIG. 4), and may include a gel 160 stored inside a pouch 162 to extract heat or provide heat to the body part 200 or the article. In an embodiment, the distance ‘d’ may be between 0.2 inches to 0.8 inches. In an embodiment, the gel 160 may include one of diethylene glycol, ethylene glycol, hydroxyethyl cellulose, sodium polyacrylate, and vinyl-coated silica gel. In an embodiment, the gel 160 may be made of a material that freezes and solidifies at zero degrees Celsius, for example.

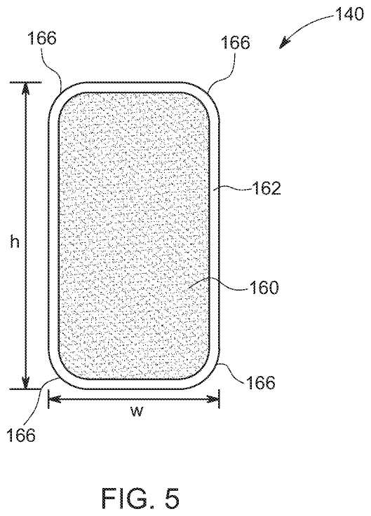

Further, in an embodiment, the pouch 162 may include a rectangular shape (best shown in FIG. 5) having round corners 166. Although the rectangular pouch 162 is contemplated, it may be envisioned that the pouch 162 may include any shape, such as, but not limited to, a circular shape, an elliptical shape, a square shape, a polygonal shape, or any other shape known in the art, and may further have right angled or otherwise non-rounded corners 166. As the pouch 162 for retaining the gel 160 has a rectangular shape, each gel cell 140 may include a rectangular shape having dimensions, such as, a width ‘w’, a height or length ‘h’, and a thickness T (shown in FIG. 3). In an embodiment, the dimension of the gel cell 140, and hence, the pouch 162, are complimentary to the inner dimensions of the pocket 134. In an embodiment, the width ‘w’ may be between 1 inch and 3 inches. Further, the height ‘h’ may be between 2 inches to 8 inches. Also, the thickness may be between 0.02 inches and 1 inch.

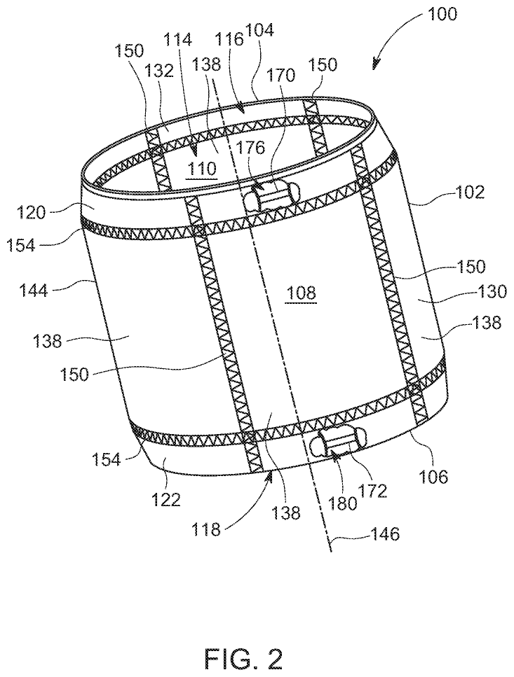

Further, the device 100 may include a first elastic cord 170 disposed proximate to the first edge 104 and attached to the first terminal circumference 120 and a second elastic cord 172 disposed proximate to the second edge 106 and attached to the second terminal circumference 122. In certain implementations, the device 100 may include a first elongated channel 176 (shown in FIG. 2) defined at the first edge 104 to receive the first elastic cord 170, and a second elongated channel 180 (shown in FIG. 2) defined at the second edge 106 to receive the second elastic cord 172. The first elongated channel 176 may extend circularly around the central axis 146 along the entire first terminal circumference 120, while the second elongated channel 180 may extend circularly around the central axis 146 along the entire second terminal circumference 122. In an embodiment, the first elastic cord 170 facilitates in varying a size of the first access opening 116 or the first terminal circumference 120, while the second elastic cord 172 facilitates in varying a size of the second access opening 118 or the second terminal circumference 122. Accordingly, when unstretched, a diameter of the first terminal circumference 120 or the first access opening 116 may be smaller than a diameter of the elongated cavity 114 at a location between the first edge 104 and the second edge 106, for example, at a mid-point between the first edge 104 and the second edge 106. In an embodiment, the diameter of the first terminal circumference 120, when unstretched, may be 5 percent to 15 percent smaller than the diameter at the mid-point. Similarly, a diameter of the second terminal circumference 122 or the second access opening 118, when unstretched, may be smaller than a diameter of the elongated cavity 114 at a location between the first edge 104 and the second edge 106, for example, at the mid-point. In an embodiment, the diameter of the second terminal circumference 122, when unstretched, may be 5 percent to 15 percent smaller than the diameter at the mid-point. As the size of the first terminal circumference 120 and the size of the second terminal circumference 122 can vary due to the presence of the elastic cords 170, 172, the device 100 abuts the surface of the body part 200 and securely holds the body part 200 when the device is worn by the wearer. Accordingly, the device 100 is retained with the body part 200 or the article without use of any fastener, such as, Velcro®, straps, etc. Further, elastic fabric of the inner fabric layer 132 and the outer fabric layer 130 facilitates and ensures an abutment of the inner surface 110 of the device 100 with the body part 200 or the article, thereby ensuring a efficient and even heat transfer between the gel cells 140 (i.e. the device 100) and the body part 200 or the article.

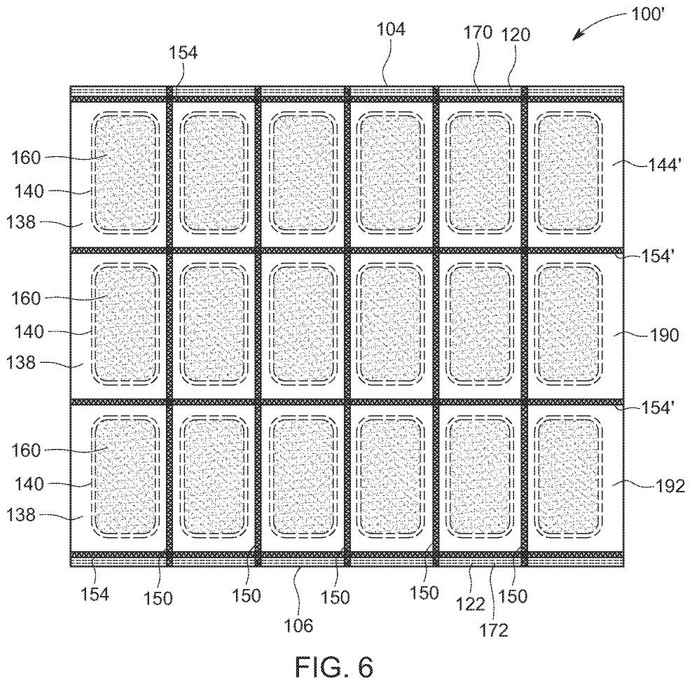

Referring to FIG. 6, a device 100′ is shown according to an embodiment of the disclosure. The device 100′ is similar to the device 100 except that the device 100′ includes a plurality of panel rows, for example, a first panel row 144′, a second panel row 190, and a third panel row 192, and each of the panel rows 144′, 190, 192 has six panels. Further, the elements of the device 100′ having similar constructional and structural aspects as that of the elements of the device 100 will have same reference numerals. The second panel row 190 is disposed spaced apart from the first panel row 144′ may be located adjacent and below the first panel row 144′. In this manner, the first panel row 144′ is disposed between the first terminal circumference 120 or the first edge 104 and the second panel row 190. Therefore, the second panel row 190 is adjacent to the first panel row 144′ and separates/spaces the first panel row 144′ from the second terminal circumference 122. Further, the third panel row 192 is disposed spaced apart from the second panel row 190 and may be disposed between the second panel row 190 and the second terminal circumference 122 or the second edge 106. Further, as shown, the adjacent panel rows are separated from each other by the stretch stitches 154′. The stretch stitches 154′ are similar in construction and formation to the second stretch stitches 154.

A method for manufacturing the device 100 is now described. It may be appreciated that the device 100′ may similarly be manufactured. The method includes providing an inner fabric layer 132. The inner fabric layer 132 is provided by cutting the elastic fabric into appropriate or suitable size. Thereafter, the gel cells 140 are positioned over the inner fabric layer 132. In an embodiment, the gel cells 140 are arrayed linearly and are placed/positioned spaced apart from each other over the inner fabric layer 132. In this manner the gel cells 140 are positioned in a single row. Thereafter, the outer fabric layer 130 is overlayed over the gel cells 140 and the inner fabric layer 132 such that the edges of the outer fabric layer 130 and edges of the inner fabric layer 132 abut each other. Subsequently, the inner fabric layer 132 and the outer fabric layer 130 are attached along the lateral edges by using second stretch stitches 154. Further, the inner fabric layer 132 and the outer fabric layer are attached/stitched together by the first stretch stitches 150 extending longitudinally and disposed between the adjacent gel cells 140. Thereafter, the longitudinal edges of the inner fabric layer 132 and the outer fabric layer 130 are attached together using the first stretch stitch 150 such that a ring or cylindrical shape is formed. Upon forming the ring, the first elastic cord 170 is attached to the first terminal circumference 120, while the second elastic cord 172 is attached to the second terminal circumference 122. In this manner, the device 100 is manufactured.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense

### LIST OF ELEMENTS

100 flexible thermal device
100′ flexible thermal device
102 elastic fabric ring
104 first edge
106 second edge
108 outer surface
110 inner surface
114 elongated cavity
116 first access opening
118 second access opening
120 first terminal circumference
122 second terminal circumference
130 outer fabric layer
132 inner fabric layer
134 pocket
138 panel
140 gel cell
144 first panel row
144′ first panel row
146 central axis
150 first stretch stitch
154 second stretch stitch
154′ stretch stitch
160 gel
162 pouch
166 round corner
170 first elastic cord
172 second elastic cord
176 first elongated channel
180 second elongated channel
190 second panel row
192 third panel row
200 body part
202 arm

What is claimed is:

1. A flexible thermal device comprising:
an elastic fabric ring having an inner fabric layer and an outer fabric layer and first terminal circumference at a first edge and a second terminal circumference at an opposite second edge;
a plurality of panels defined between the inner fabric layer and the outer fabric layer creating a first panel row; and
a plurality of gel cells being retained in the plurality of panels in the first panel row;
wherein the panels are bordered by stretch stitches joining the inner fabric layer and the outer fabric layer.

2. The device of claim 1 wherein the inner fabric layer and the outer fabric layer are formed of fabric that is elastic in four different directions.

3. The device of claim 1 wherein the inner fabric layer and the outer fabric layer are formed of fabric that can be stretched 25% longer than an unstretched length, and returns to the original length when released.

4. The device of claim 1 wherein the first panel row has at least four panels.

5. The device of claim 1, further comprising a second panel row adjacent to the first panel row.

6. The device of claim 5 wherein the second panel row is adjacent to the first panel row and spacing the first panel row from the second terminal circumference.

7. The device of claim 5, further comprising a third panel row adjacent to the second panel row, wherein the second panel row spaces the first panel row from the third panel row.

8. The device of claim 1 wherein the first, second, and third panel row each contain six panels.

9. The device of claim 1 wherein the first panel row contains an even number of panels.

10. The device of claim 1 wherein each panel has only one gel cell retained within.

11. The device of claim 1 wherein the stretch stitch is one of a Triple Straight Stitch, a Zigzag Stitch, a Three Step Zigzag stitch, a Lightning Bolt Stitch, a Honeycomb Stitch, an Overlock/Overedge Stitch, a Feather Stitch, a twin needle stitch, blind hem stretch stitch, and the stitch is with substantially non-elastic thread.

12. The device of claim 1 wherein the stretch stitch is a strait stitch with elastic thread.

13. The device of claim 1 wherein a first elastic cord is attached to the first terminal circumference and a second elastic cord is attached to the second terminal circumference.

14. The device of claim 1 wherein the gel cells contain one of diethylene glycol, ethylene glycol, hydroxyethyl cellulose, sodium polyacrylate, and vinyl-coated silica gel.

15. The device of claim 1 wherein the gel cells freeze solid at 0 degrees Celsius.

16. The device of claim 1 wherein the gel cells measure between 1 and 3 inches in width, 2 and 8 inches in height, and between 0.02 and 1 inch in thickness.

17. The device of claim 1 wherein adjacent gel cells are spaced between 0.2 and 0.8 inches from one another.

18. The device of claim 1 wherein the inner fabric layer and an outer fabric layer are one of spandex or spandex blended with a second fabric.

19. The device of claim 1 wherein the first and the second terminal circumference have a relaxed diameter 5% to 15% smaller than a diameter of the device at a mid-point between the first and the second terminal circumference.

20. A flexible thermal device comprising:
an elastic fabric ring having an inner fabric layer and an outer fabric layer and first terminal circumference at a first edge and a second terminal circumference at an opposite second edge;
a plurality of panels defined between the inner fabric layer and the outer fabric layer creating a first panel row, the panels being bordered by stretch stitches joining the inner fabric layer and the outer fabric layer;
a second panel row adjacent to the first panel row, the second panel row being adjacent to the first panel row and spacing the first panel row from the second terminal circumference;
the first and the second panel row each contain six panels;
a plurality of gel cells being retained in the plurality of panels in the first and the second panel row, with each panel having only one gel cell retained within;
the gel cells measure between 1 and 3 inches in width, 2 and 8 inches in height, and between 0.02 and 1 inch in thickness;
the inner fabric layer and the outer fabric layer are formed of fabric that is elastic in four different directions;
the inner fabric layer and the outer fabric layer are formed of fabric that can be stretched 25% longer than an unstretched length in four directions, and returns to the original length when released;
the stretch stitch being one of a Triple Straight Stitch, a Zigzag Stitch, a Three Step Zigzag stitch, a Lightning Bolt Stitch, a Honeycomb Stitch, an Overlock/Overedge Stitch, a Feather Stitch, a twin needle stitch, blind hem stretch stitch, and the stitch is with substantially non-elastic thread or being a strait stitch with substantially elastic thread, or some combination thereof;
a first elastic cord being attached to the first terminal circumference and a second elastic cord being attached to the second terminal circumference;
the gel cells containing one of diethylene glycol, ethylene glycol, hydroxyethyl cellulose, sodium polyacrylate, and vinyl-coated silica gel, or some combination thereof;
the gel cells freezing solid when left for 24 hours at one of 2 degrees Celsius, 0 degrees Celsius, −5 degrees Celsius, and −10 degrees Celsius;
adjacent gel cells being spaced between 0.2 and 0.8 inches from one another;
the inner fabric layer and an outer fabric layer are one of spandex or spandex blended with a second fabric; and
the first and the second terminal circumference having a relaxed diameter 5% to 15% smaller than a diameter of the device at a mid-point between the first and the second terminal circumference.

21. A method of treating an injury comprising:
inserting an injured body part within the flexible thermal device of claim 1, wherein the gel cells of the flexible thermal device are one of above 38, 40, 42, 45, or 48 degrees Celsius and below 9.5, 5, 2, or 0 degrees Celsius for a period of at least 15 minutes.
